(19) United States
(12) Patent Application Publication
(10) Pub. No.: US 2026/0167243 A1
(43) Pub. Date: Jun. 18, 2026

(54) ELECTRIC-ASSISTED SLIDING BOARD

(71) Applicant: Wonderfold(Guangzhou) Children's Products Co., Ltd, Guangzhou (CN)

(72) Inventor: Sze Wah Sarah Ng, IRWINDALE, CA (US)

(21) Appl. No.: 19/368,012

(22) Filed: Oct. 24, 2025

(30) Foreign Application Priority Data
Oct. 29, 2024 (CN) 202422638119.3

(51) Int. Cl. B62B 7/08; A61B 17/64; B62B 9/20

(52) U.S. Cl. CPC B62B 7/08; A61B 17/6466; B62B 9/20

(57) ABSTRACT

An electric-assisted sliding board adapted to a trolley, where the trolley includes a supporting frame arranged at a rear end thereof. The electric-assisted sliding board includes a board body, a sliding wheel, a drive mechanism and a connecting assembly. The drive mechanism is arranged on the board body. The sliding wheel is connected to the drive mechanism. The drive mechanism is configured to drive the sliding wheel to rotate, thereby driving the sliding board to move forward. The connecting assembly is connected to the supporting frame to allow the electric-assisted sliding board to push the trolley forward. The connecting assembly is arranged at a front end of the board body, and is connected to the supporting frame of the trolley, such that the electric-assisted sliding board can push the trolley forward.

### CROSS-REFERENCE TO RELATED APPLICATIONS

This application claims the benefit of priority from Chinese Patent Application No. 202422638119.3, filed on Oct. 29, 2024. The content of the aforementioned application, including any intervening amendments thereto, is incorporated herein by reference in its entirety.

### TECHNICAL FIELD

This application relates to trolleys, and more particularly to an electric-assisted sliding board.

### BACKGROUND

Baby strollers have been increasingly popularized, and are often carried for outdoor activities. When unfolded, they can provide a seat for infants and young children, thus reducing the burden on caregivers.

The existing baby strollers include a frame, a front wheel assembly arranged under a front portion of the frame, and a rear wheel assembly arranged under a rear portion of the frame. The movement of the baby stroller primarily relies on the pushing force applied to a handlebar of the frame, combined with the rotation of front and rear wheels. However, the prolonged pushing or poor road conditions will lead to excessive labor consumption.

### SUMMARY

An object of the present disclosure is to provide an electric-assisted sliding board to address the technical problem in the prior art that it is relatively strenuous to push a baby stroller over prolonged period or on an uneven road surface.

In order to achieve the above object, the following technical solutions are adopted.

An electric-assisted sliding board for a trolley, the trolley comprising a supporting frame arranged at a rear end of the trolley, and the electric-assisted sliding board comprising:
a board body;
a sliding wheel;
a drive mechanism; and
a connecting assembly;
wherein the drive mechanism is arranged on the board body;
the sliding wheel is connected to the drive mechanism;
the drive mechanism is configured to drive the sliding wheel to rotate, so as to drive the electric-assisted sliding board to move; and
the connecting assembly is connected to the supporting frame to allow the electric-assisted sliding board to push the trolley to move forward.

In an embodiment, the connecting assembly comprises a fixing clamp, a connecting part, a bearing and a first fastener; the board body is provided with a first mounting hole; the bearing is provided with a center hole; a first end of the connecting part is connected to the fixing clamp; and a second end of the connecting part is configured to pass through the first mounting hole and the center hole of the bearing sequentially to be connected to the first fastener to secure the board body between the fixing clamp and the bearing.

In an embodiment, the connecting part comprises a first cylindrical segment and a second cylindrical segment opposite to each other; and the first cylindrical segment is larger than the second cylindrical segment in terms of diameter; and
the fixing clamp is provided with a first groove; the first cylindrical segment is provided with a second groove extending along an axial direction of the first cylindrical segment; the second cylindrical segment is fixedly mounted in the first groove; and the first fastener is fixedly mounted in the second groove.

In an embodiment, the first groove and the second groove are each provided with an internal thread, and the second cylindrical segment and the first fastener are each provided with an external thread; the second cylindrical segment is threadedly connected to the fixing clamp; and the first fastener is threadedly connected to the first cylindrical segment.

In an embodiment, the connecting assembly further comprises a second fastener; a lifting hug is arranged on an outer periphery of the bearing, and is provided with a through hole; the board body is provided with a second mounting hole corresponding to the through hole; and the second fastener is configured to pass through the through hole and the second mounting hole sequentially to fixedly connect the board body to the bearing.

In an embodiment, the connecting part is in interference fit with the center hole of the bearing.

In an embodiment, the fixing clamp comprises a first clamping portion, a second clamping portion, a handle and a pin shaft; the first clamping portion is hinged to the second clamping portion; and the handle is fixedly connected to the pin shaft; and
the pin shaft is provided with a first threaded segment and a second threaded segment; a rotation direction of the first threaded segment is opposite to that of the second threaded segment; the first threaded segment is configured to fit the first clamping portion; the second threaded segment is configured to fit the second clamping portion; and the handle is configured to drive the pin shaft to rotate to allow the first clamping portion and the second clamping portion to move toward or away from each other.

In an embodiment, the fixing clamp further comprises a first post and a second post; the first clamping portion is provided with a first curved groove configured for mounting the first post; and the second clamping portion is provided with a second curved groove configured for mounting the second post; and
the first post, the second post, the first clamping portion and the second clamping portion are each provided with an insertion hole to allow the pin shaft to pass through; an inner wall of the insertion hole of the first post is threadedly connected to the first threaded segment; and an inner wall of the insertion hole of the second post is threadedly connected to the second threaded segment.

In an embodiment, a side of the first clamping portion facing toward the second clamping portion and a side of the second clamping portion facing toward the first clamping portion are each provided with an anti-slip rubber pad.

In an embodiment, the connecting assembly comprises a fixing clamp, a first connecting part, a second connecting part, a bearing and a fastener; the board body is provided with a mounting hole; the bearing is provided with a center hole; the second connecting part is connected to the fixing clamp; a first end of the first connecting part is connected to the second connecting part; and a second end of the first connecting part is configured to pass through the mounting hole and the center hole of the bearing sequentially to be connected to the fastener to secure the board body between the fixing clamp and the bearing.

Regarding the electric-assisted sliding board provided herein, the connecting assembly is arranged at a front end of the board body, and is adapted to be connected to the supporting frame of the trolley, such that the electric-assisted sliding board can push the trolley forward to move forward. The user can stand and ride on the board body during operation of the electric-assisted sliding board, which can effectively reduce the labor consumption and enhance the use comfort.

### BRIEF DESCRIPTION OF THE DRAWINGS

In order to make technical solutions in the embodiments of the present disclosure or in the prior art clearer, the accompanying figures needed in the description of the embodiments of the present disclosure or the prior art will be briefly introduced below. Obviously, presented in the accompanying figures are merely some embodiments of the present disclosure. Other accompanying figures can be obtained by those skilled in the art based on these accompanying figures without paying creative efforts.

FIG. 1 schematically shows an electric-assisted sliding board according to an embodiment of the present disclosure;

FIG. 2 schematically shows the electric-assisted sliding board according to the embodiment of the present disclosure from another perspective;

FIG. 3 schematically shows a board body according to an embodiment of the present disclosure;

FIG. 4 schematically shows a connecting assembly according to an embodiment of the present disclosure;

FIG. 5 is a sectional view of the connecting assembly according to the embodiment of the present disclosure;

FIG. 6 schematically shows a first connecting part according to an embodiment of the present disclosure;

FIG. 7 schematically shows a bearing according to an embodiment of the present disclosure;

FIG. 8 schematically shows a fixing clamp according to an embodiment of the present disclosure;

FIG. 9 is an exploded view of the fixing clamp according to the embodiment of the present disclosure;

FIG. 10 schematically shows an electric-assisted sliding board according to another embodiment of the present disclosure;

FIG. 11 schematically shows a connecting assembly according to another embodiment of the present disclosure;

FIG. 12 is a sectional view of the connecting assembly according to another embodiment of the present disclosure; and

FIG. 13 schematically shows a second connecting part according to an embodiment of the present disclosure.

In the figures: 10—board body; 11—first mounting hole; 12—second mounting hole; 13—decoration plate; 20—sliding wheel; 30—drive mechanism; 40—connecting assembly; 41—fixing clamp; 411—first clamping portion; 4111—first curved groove; 4112-insertion hole; 4113—anti-slip rubber pad; 412—second clamping portion; 4121—first groove; 4122—second curved groove; 413—handle; 414—pin shaft; 414a—first threaded segment; 414b—second threaded segment; 415—clamping area; 416—hinge shaft; 417—first post; 418—second post; 419—spring; 42—first connecting part; 421—first cylindrical segment; 422—second cylindrical segment; 423—second groove; 43—bearing; 431—center hole; 432—lifting lug; 433—through hole; 44—first fastener; 45—second fastener; 46—second connecting part; and 461—third groove.

The objective implementation, functional features and advantages of the present disclosure will be further described below in conjunction with the embodiments and the accompanying figures.

### DETAILED DESCRIPTION OF EMBODIMENTS

The technical solutions of the embodiments in this disclosure will be described clearly and completely with reference to the accompanying figures. Obviously, the described embodiments are merely some embodiments of the present disclosure, instead of all embodiments of the present disclosure. Other embodiments obtained by those skilled in the art based on these embodiments without paying creative efforts shall fall within the scope of the disclosure defined by the appended claims.

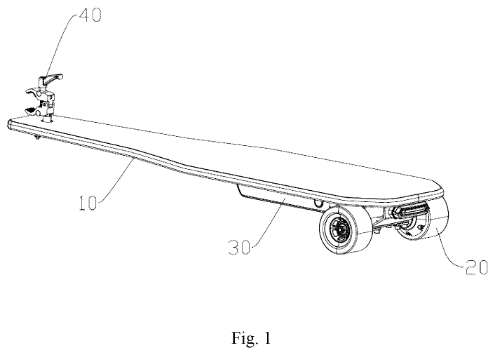

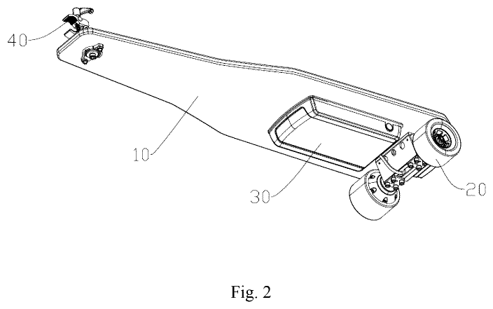

According to an embodiment in FIGS. 1-2, an electric-assisted sliding board includes a board body 10, a sliding wheel 20, a drive mechanism 30 and a connecting assembly 40. The sliding wheel 20 is arranged on a rear end of the board body 10. The drive mechanism 30 is arranged on the board body 10, and is connected to the sliding wheel 20 to drive the sliding wheel 20 to rotate, so as to drive the electric-assisted sliding board to move.

The connecting assembly 40 is arranged on a front end of the board body 10, and is configured to connect the electric-assisted sliding board with a transportation device such as a baby stroller to push the transportation device to move via the electric-assisted sliding board. The user can stand and ride on the board body 10 during operation of the electric-assisted sliding board, which can effectively reduce the labor consumption and enhance the use comfort.

It should be understood that most of the existing baby strollers don't have power systems, such that the prolonged pushing or poor road conditions will lead to excessive labor consumption. In this case, by connecting the electric-assisted sliding board to the transportation device such as the baby stroller, the user can stand and ride on the board body 10. Moreover, the drive mechanism 30 is configured to drive the electric-assisted sliding board to move forward, so as to drive the baby stroller to move forward. It can effectively reduce the labor consumption and enhance the use comfort.

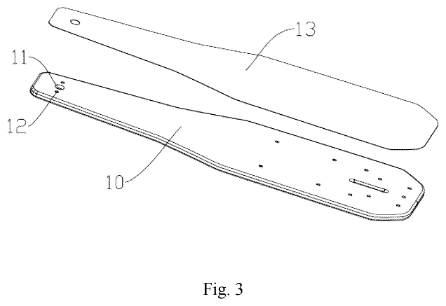

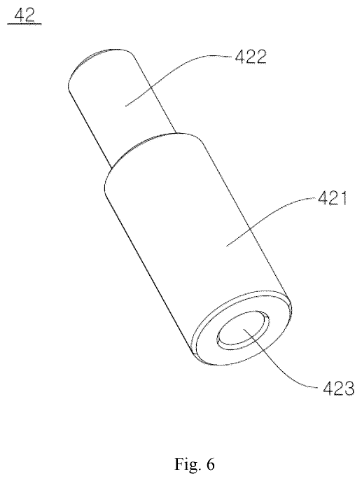

According to some embodiments in FIGS. 3-7, the board body 10 is provided with a first mounting hole 11. The connecting assembly 40 includes a fixing clamp 41, a first connecting part 42, a bearing 43 and a first fastener 44. The bearing 43 is provided with a center hole 431. The first connecting part 42 is configured to pass through the first mounting hole 11 and the center hole 431 of the bearing 43 sequentially. The first connecting part 42 is provided with a first end and a second end opposite to each other. The first end of the first connecting part 42 is connected to the fixing clamp 41, and the second end of the first connecting part 42 is connected to the first fastener 44 to secure the bearing 43 and the board body 10 between the fixing clamp 41 and the first fastener 44. After assembling the board body 10 and the connecting assembly 40, the bearing 43 is arranged on a lower portion of the board body 10, and the fixing clamp 41 is arranged on an upper portion of the board body 10, so as to facilitate a connection between the fixing clamp 41 and the transportation device such as the baby stroller.

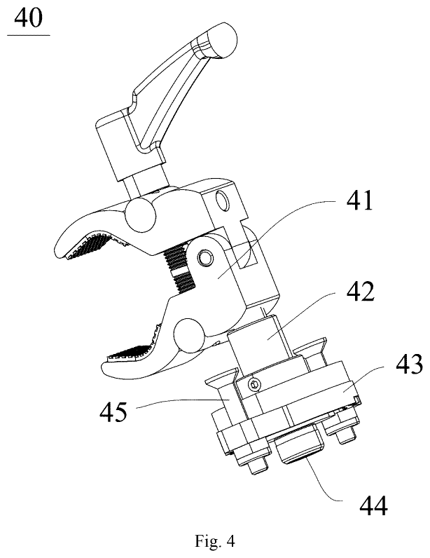

According to an embodiment in FIGS. 4-6, the first connecting part 42 includes a first cylindrical segment 421 and a second cylindrical segment 422 opposite to each other. The first cylindrical segment 421 is larger than the second cylindrical segment 422 in terms of diameter. The second cylindrical segment 422 is provided with an external thread, and the fixing clamp 41 is provided with a first groove 4121. An internal thread fitting the external thread of the second cylindrical segment 422 is provided inside the first groove 4121. The second cylindrical segment 422 is in interference fit with the first groove 4121. In other words, the external thread of the second cylindrical segment 422 is in interference fit with the internal thread of the first groove 4121 to fastenedly connect the first connecting part 42 to the fixing clamp 41. The first cylindrical segment 421 is provided with a second groove 423 extending along an axial direction. An internal thread is provided inside the second groove 423. The first fastener 44 is a bolt. After the connecting part 42 passes through the first mounting hole 11 and the center hole 413 of the bearing 43, the first fastener 44 is in interference fit with the internal thread of the second groove 423 to secure the bearing 43 and the board body 10 between the fixing clamp 41 and the first fastener 44.

In some embodiments, referring to FIGS. 3-4 and 7, in order to secure the board body 10 and the bearing 43, and improve a stability performance of the electric-assisted sliding board, the connecting assembly 40 further includes a second fastener 45. A plurality of lifting lugs 432 spaced apart from each other are arranged on an outer periphery of the bearing 43. A through hole 433 is arranged on each of the plurality of lifting lugs 432. A second mounting hole 12 corresponding to the through hole 433 is arranged on the board body 10. The second fastener 45 is configured to fit the though hole 433 and the second mounting hole 12, respectively, such that the board body 10 is fixedly connected to the bearing 43.

In some embodiments, the second fastener 45 includes a nut and a bolt. The nut is configured to pass through the second mounting hole 12 and the through hole 433 sequentially to be threadedly connected to the bolt, such that the board body 10 is fixedly connected to the bearing 43.

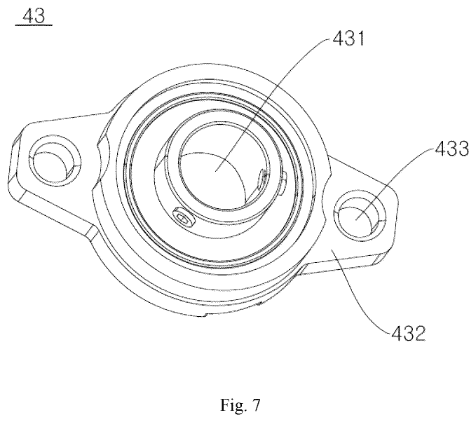

According to an embodiment in FIG. 7, the number of the lifting lug 432 is two. The two lifting lugs 432 are symmetrically arranged on the bearing 43. The two lifting lugs 432 are each provided with the through hole 433. The number of the second mounting hole 12 is two, and the number of the second fastener 45 is two. Each of the two second mounting hole 12 is correspondingly arranged with each of the two through holes 433, such that the board body 10 is connected to the bearing 43 via the second fastener 45. In this case, the two lifting lugs 432 are symmetrically arranged on the bearing 43 to achieve uniform force distribution thereon, further improving a connection strength between the bearing 43 and the board body 10.

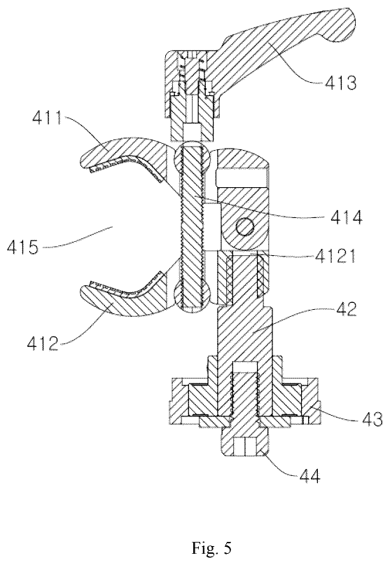

According to an embodiment in FIG. 5, an outer surface of the first connecting part 42 is configured to abut against an inner surface of the bearing 43. In other words, the first cylindrical segment 421 of the first connecting part 42 is in interference fit with the center hole 431 of the bearing 43, such that an axial displacement of the bearing 43 and the board body 10 on the first connecting part 42 is decreased to improve the stability performance of the electric-assisted sliding board. An angel of the fixing clamp 41 is changed by rotating the bearing 43 to connect the fixing clamp 41 to various transportation devices, so as to improve a versatility of the connecting assembly 40 and satisfy diverse requirements.

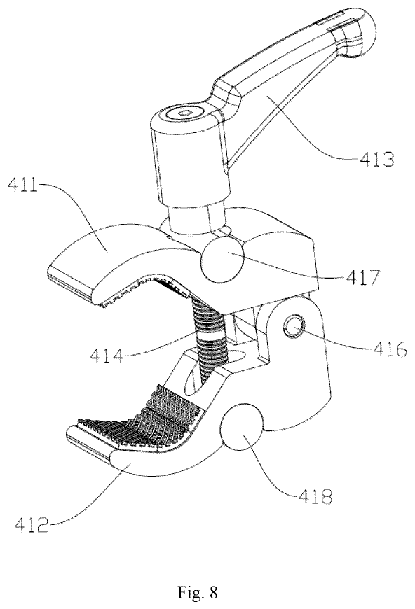

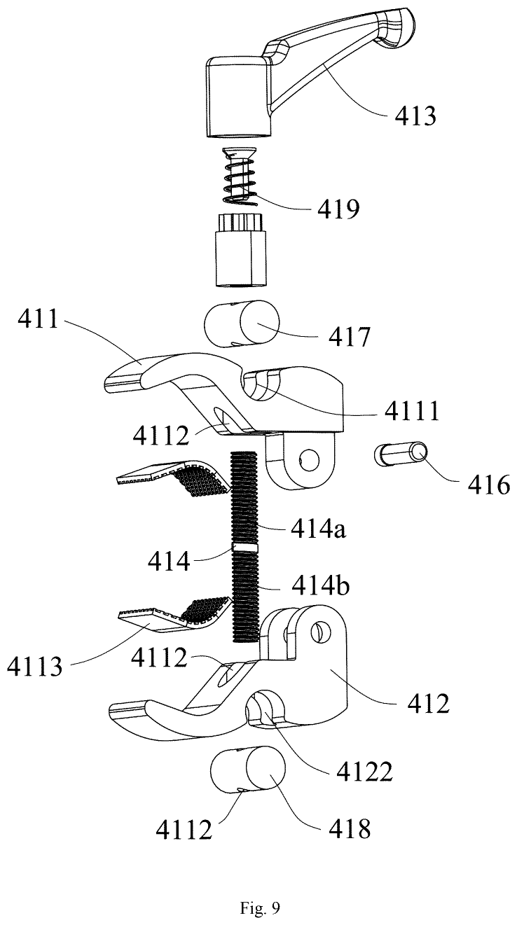

According to embodiments in FIGS. 8-9, the fixing clamp 41 includes a first clamping portion 411, a second clamping portion 412, a handle 413 and a pin shaft 414. The first clamping portion 411 is hinged to the second clamping portion 412. The handle 413 is fixedly connected to the pin shaft 414. The pin shaft 414 is provided with a first threaded segment 414a and a second threaded segment 414b. A rotation direction of the first threaded segment 414a is opposite to that of the second threaded segment 414b. The first threaded segment 414a is configured to fit the first clamping portion 411. The second threaded segment 414b is configured to fit the second clamping portion 412. The handle 413 is configured to drive the pin shaft 414 to rotate to allow the first clamping portion 411 to move toward or away from the second clamping portion 412.

It should be understood that the first clamping portion 411 is configured to enclose with the second clamping portion 412 to form a clamping area 415. A supporting frame is usually arranged on a rear bottom of the trolley. The supporting frame is an axle or a frame of the trolley. The fixing clamp 41 is connected to the supporting frame.

In some embodiments, a first end of the first clamping portion 411 is connected to a first end of the second clamping portion 412 via a hinge shaft 416, such that the first clamping portion 411 and the second clamping portion 412 are configured to rotate relatively to each other. A second end of the first clamping portion 411 and a second of the second clamping portion 412 are each configured as a curved end. The curved end of the first clamping portion 411 and the curved end of the second clamping portion 412 are arranged opposite to each other to enclose and form the clamping area 415. The clamping area 415 is configured to be curved to more stably connect the fixing clamp 41 to the supporting frame of the trolley. When the trolley moves forward, the electric-assisted sliding board is less likely to fall off, enhancing the overall safety performance.

According to some embodiments in FIGS. 8-9, the fixing clamp 41 further includes a first post 417 and a second post 418. The first clamping portion 411 is provided with a first curved groove 4111 configured for mounting the first post 417. The first post 417 is mounted in the first curved groove 4111. The second clamping portion 412 is provided with a second curved groove 4122 configured for mounting the second post 418. The second post 418 is mounted in the second curved groove 4122. The first post 417, the second post 418, the first clamping portion 411 and the second clamping portion 412 are each provided with an insertion hole 4112 to allow the pin shaft 414 to pass through. The insertion hole 4112 of the first post 417 and the insertion hole 4112 of the second post 418 are each provided with an internal thread. The internal thread of the insertion hole 4112 of the first post 417 is threadedly connected to the first thread segment 414a of the pin shaft 414. The internal thread of the insertion hole 4112 of the second post 418 is threadedly connected to the second thread segment 414b of the pin shaft 414.

The rotation direction of the first threaded segment 414a is opposite to that of the second threaded segment 414b, such that when the pin shaft 414 is rotated via the handle 413, the first post 417 and the second post 418 are configured to move toward or away from each other. The first post 417 is fixedly connected to the first clamping portion 411, and the second post 418 is fixedly connected to the second clamping portion 412 to allow the first clamping portion 411 and the second clamping portion 412 to move toward or away from each other under an action of the first post 417 and the second post 418, thereby changing a size of the clamping area 415 to allow the fixing clamp 41 to fit the supporting frame of different sizes, and improving the versatility of the electric-assisted sliding board to generalize the applicational range. Moreover, the size of the clamping area 415 is adjustable to ensure a clamping stability of the fixing clamp 41 and reduce the risk of accidents.

According to some embodiments in FIG. 5 and FIG. 9, a spring 419 is arranged between the handle 413 and the pin shaft 414 to enable an extension and compress of the spring 419 to achieve an opening and closing of the fixing clamp 41.

In some embodiments, when the handle 413 is tightly gripped, the spring 419 is compressed under force, causing the first clamping portion 411 and the second clamping portion 412 to close. When the handle 413 is released, the spring 419 extends, allowing the first clamping portion 411 and the second clamping portion 412 to open.

Referring to FIGS. 8-9, the first clamping portion 411 and the second clamping portion 412 are each provided with an anti-slip rubber pad 4113. A side of the first clamping portion 411 facing toward the second clamping portion 412 and a side of the second clamping portion 412 facing toward the first clamping portion 411 are each provided with the anti-slip rubber pad 4113. The anti-slip rubber pad 4113 is adhesively connected to the first clamping portion 411 and the second clamping portion 412, respectively. In this case, a frictional force of the fixing clamp 41 is increased via the anti-slip rubber pad 4113 to stably connect it to the supporting frame of the trolley.

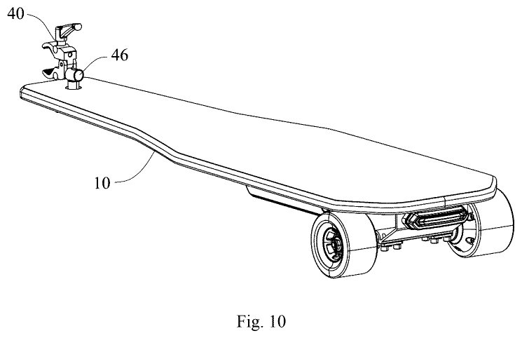

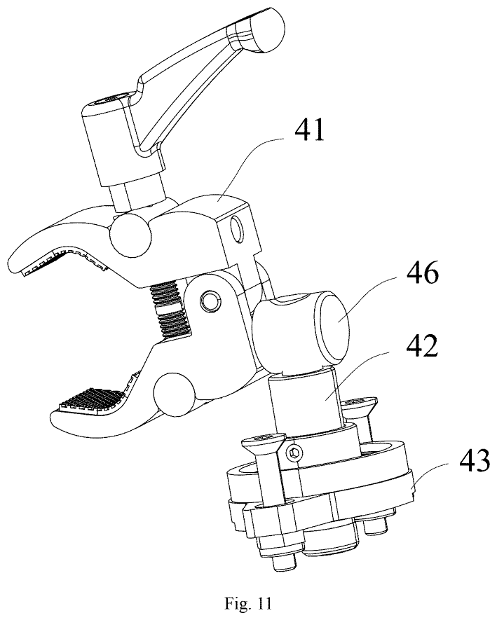

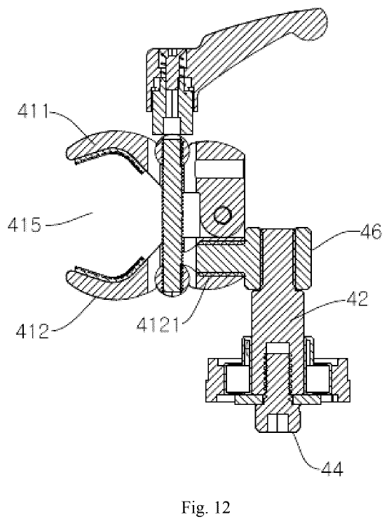

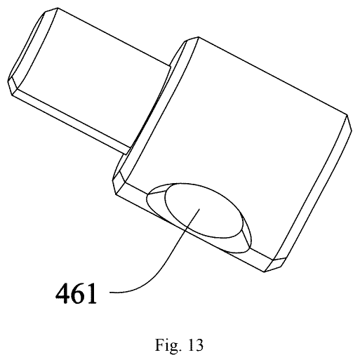

According to an embodiment in FIGS. 10-13, the difference between this embodiment and the aforementioned embodiments lies where the connecting assembly 40 further includes a second connecting part 46 arranged on the fixing clamp 41, and the first connecting part 42 is arranged on the second connecting part 46.

In this embodiment, a position of the first groove 4121 is different from that of the first groove 4121 in the aforementioned embodiments. In this case, the first groove 4121 is arranged on a side of the fixing clamp 41. The second connecting part 46 includes a first cylindrical segment and a second cylindrical segment opposite to each other. The first cylindrical segment is larger than the second cylindrical segment in terms of diameter. The second cylindrical segment of the second connecting part 46 is arranged in the first groove 4121, such that the second connecting part 46 is threadedly connected to the fixing clamp 41. A side of the first cylindrical segment of the second connecting part 46 is provided with a third groove 461. An inner wall of the third groove 461 is configured as a threaded shape. The second cylindrical segment 422 of the first connecting part 42 is arranged in the third groove 461, such that the first connecting part 42 is threadedly connected to the second connecting part 46 to ensure a connection strength between the first connecting part 42 and the second connecting part 46. A distance between the first connecting part 42 and the second clamping portion 412 is increased via the second connecting part 46, which is convenient for users to adjust a length, so as to fit trolleys of different shapes and sizes.

In an embodiment, the drive mechanism 30 includes a battery, where the battery is a rechargeable battery or a dry battery. The battery is configured to provide a power source for an operation of the electric-assisted sliding board.

In an embodiment, a decoration plate 13 is arranged on a side of the board body 10 facing away from drive mechanism 30. The decoration plate 13 is configured to fit a shape of the board body 10. The decoration plate 13 is adhesively connected to the board body 10. The mounting holes of the board body 10 are covered through the decoration plate 13 to improve an aesthetic performance of the electric-assisted sliding board.

In the above embodiments, the description of each of the above embodiments has different focuses. An embodiment without detailed descriptions can refer to the relevant descriptions of other embodiments.

In the description of the present disclosure, the terms “first” and “second” are only for descriptive purposes, and should not be understood as indicating or implying the relative importance or the quantity of the technical features involved. Therefore, features defined by “first” or “second” may explicitly or implicitly indicate the inclusion of at least one of such features.

The electric-assisted sliding board disclosed herein has been described in detail above. Described above are merely some embodiments of the present disclosure for illustrating the principle and implementation method of this disclosure. The descriptions of the above embodiments are for better understanding the implementation method and the core concept of the present disclosure. For those skilled in the art, various modifications made without departing from the spirit of the disclosure shall fall within the scope of the disclosure defined by the appended claims.

What is claimed is:

1. An electric-assisted sliding board for a trolley, the trolley comprising a supporting frame arranged at a rear end of the trolley, and the electric-assisted sliding board comprising:
a board body;
a sliding wheel;
a drive mechanism; and
a connecting assembly;
wherein
the drive mechanism is arranged on the board body;
the sliding wheel is connected to the drive mechanism;
the drive mechanism is configured to drive the sliding wheel to rotate, so as to drive the electric-assisted sliding board to move; and
the connecting assembly is connected to the supporting frame to allow the electric-assisted sliding board to push the trolley to move forward.

2. The electric-assisted sliding board of claim 1, wherein the connecting assembly comprises a fixing clamp, a connecting part, a bearing and a first fastener; the board body is provided with a first mounting hole; the bearing is provided with a center hole; a first end of the connecting part is connected to the fixing clamp; and a second end of the connecting part is configured to pass through the first mounting hole and the center hole of the bearing sequentially to be connected to the first fastener to secure the board body between the fixing clamp and the bearing.

3. The electric-assisted sliding board of claim 2, wherein the connecting part comprises a first cylindrical segment and a second cylindrical segment opposite to each other; and the first cylindrical segment is larger than the second cylindrical segment in terms of diameter; and
the fixing clamp is provided with a first groove; the first cylindrical segment is provided with a second groove extending along an axial direction of the first cylindrical segment; the second cylindrical segment is fixedly mounted in the first groove; and the first fastener is fixedly mounted in the second groove.

4. The electric-assisted sliding board of claim 3, wherein the first groove and the second groove are each provided with an internal thread, and the second cylindrical segment and the first fastener are each provided with an external thread; the second cylindrical segment is threadedly connected to the fixing clamp; and the first fastener is threadedly connected to the first cylindrical segment.

5. The electric-assisted sliding board of claim 2, wherein the connecting assembly further comprises a second fastener; a lifting hug is arranged on an outer periphery of the bearing, and is provided with a through hole; the board body is provided with a second mounting hole corresponding to the through hole; and the second fastener is configured to pass through the through hole and the second mounting hole sequentially to fixedly connect the board body to the bearing.

6. The electric-assisted sliding board of claim 2, wherein the connecting part is in interference fit with the center hole of the bearing
7. The electric-assisted sliding board of claim 2, wherein the fixing clamp comprises a first clamping portion, a second clamping portion, a handle and a pin shaft; the first clamping portion is hinged to the second clamping portion; and the handle is fixedly connected to the pin shaft; and
the pin shaft is provided with a first threaded segment and a second threaded segment; a rotation direction of the first threaded segment is opposite to that of the second threaded segment; the first threaded segment is configured to fit the first clamping portion; the second threaded segment is configured to fit the second clamping portion; and the handle is configured to drive the pin shaft to rotate to allow the first clamping portion and the second clamping portion to move toward or away from each other.

8. The electric-assisted sliding board of claim 7, wherein the fixing clamp further comprises a first post and a second post; the first clamping portion is provided with a first curved groove configured for mounting the first post; and the second clamping portion is provided with a second curved groove configured for mounting the second post; and
the first post, the second post, the first clamping portion and the second clamping portion are each provided with an insertion hole to allow the pin shaft to pass through;
an inner wall of the insertion hole of the first post is threadedly connected to the first threaded segment; and an inner wall of the insertion hole of the second post is threadedly connected to the second threaded segment.

9. The electric-assisted sliding board of claim 7, wherein a side of the first clamping portion facing toward the second clamping portion and a side of the second clamping portion facing toward the first clamping portion are each provided with an anti-slip rubber pad.

10. The electric-assisted sliding board of claim 1, wherein the connecting assembly comprises a fixing clamp, a first connecting part, a second connecting part, a bearing and a fastener; the board body is provided with a mounting hole; the bearing is provided with a center hole; the second connecting part is connected to the fixing clamp; a first end of the first connecting part is connected to the second connecting part; a second end of the first connecting part is configured to pass through the mounting hole and the center hole of the bearing sequentially to be connected to the fastener to secure the board body between the fixing clamp and the bearing.
